(19) United States
(12) Patent Application Publication
(10) Pub. No.: US 2025/0152047 A1
(43) Pub. Date: May 15, 2025

(54) MEDICAL DIAGNOSTIC DEVICES AND METHODS

(71) Applicant: Intuity Medical, Inc., Fremont, CA (US)

(72) Inventors: Kelley J. LIPMAN, Livermore, CA (US); Michael F. Tomasco, Morgan Hill, CA (US); Daniel N. Brncic, Menlo Park, CA (US); David K. Hohl, Milpitas, CA (US); Paul D. Reynolds, Palo Alto, CA (US); Raul Escutia, Sunnyvale, CA (US)

(21) Appl. No.: 18/639,358

(22) Filed: Apr. 18, 2024

Related U.S. Application Data
(63) Continuation of application No. 18/067,135, filed on Dec. 16, 2022, now Pat. No. 11,986,293.
Application No. 18/067,135 is a continuation of application No. 16/536,066, filed on Aug. 8, 2019, now Pat. No. 11,553,860.
Application No. 16/536,066 is a continuation of application No. 12/457,332, filed on Jun. 8, 2009, now Pat. No. 10,383,556.
(60) Provisional application No. 61/129,148, filed on Jun. 6, 2008.

(51) Int. Cl. A61B 5/145; A61B 5/00; C12Q 1/54

(52) U.S. Cl. CPC A61B 5/14532; A61B 5/0002; C12Q 1/54

(57) ABSTRACT

A medical diagnostic device includes a wirelessly transmitted time data receiver and processor. Associated devices, methods and functionality are also described.

### FIELD

The present invention is directed to devices and methods possessing one or more of improved timekeeping abilities and functionality, and visual user interfacing capabilities.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

Currently, glucose monitors typically include a clock function that allows the device to mark each individual glucose measurement with a timestamp. As the user tests with the device over a period of time, the time data collected and the associated glucose values are used by the patient and their care givers to create charts of glucose measurements. This set of data can be used to adjust patient medication or as a tool to gauge effectiveness of current treatments.

A flaw in current systems is that they are dependent on the users to correctly set the time. Setting the time on a glucose meter is not always a straight forward task and many of the users of glucose meters are older and/or not technologically savvy. It is also very likely that many users of glucose meters are not even aware that they should be setting the time on their devices. The difficulty in correctly setting the time results in many glucose meters collecting inaccurate data that is not useable by patient or caregiver.

A problem with episodic glucose monitoring is that patients only get a snapshot of their current blood glucose. They must make treatment decisions using that snapshot, rather than using dynamic information that more accurately reflects their condition.

For example, if a patient tests and finds that their blood glucose is low, they may decide to take in some carbohydrates to raise their blood sugar. However, it may be that their glucose is already rising, in which case they may not need as much, or any carbohydrates.

Conversely, a patient may test and find that their blood glucose is at a desirable level, not realizing that their glucose is actually rapidly dropping. In this case they will probably decide not to intake any carbohydrates, when in reality they may face a hypoglycemic event soon if they don't act.

It would be desirable for the glucose meter to be able to forecast what direction and how quickly glucose levels are changing. This would allow the user to more accurately treat themselves, thus reducing the likelihood of hypoglycemic or hyperglycemic events.

In order to do such forecasting, at least two things are required: enough historical data in order to predict future trends; and accurate time-stamping information associated with those historical glucose results.

### SUMMARY OF THE INVENTION

As used herein, “body fluid” encompasses whole blood, intestinal fluid, and mixtures thereof.

As used herein “integrated device” or “integrated meter” means a device or meter that includes all components necessary to perform sampling of body fluid, transport of body fluid, quantification of an analyte, and display of the amount of analyte contained in the sample of body fluid. Exemplary integrated meters are described in: U.S. Pat. Nos. 6,540,675 and 7,004,928; U.S. Patent Application Publication Nos. US 2008/0077048, US 2007/0179404, US 2007/0083131, US 2007/0179405, US 2007/0078358, and US 2007/0078313. The entire contents of each of the above-listed documents are incorporated herein by reference.

It is to be understood that reference herein to first, second, third and fourth components (etc.) does not limit the present invention to embodiments where each of these components is physically separable from one another. For example, a single physical element of the invention may perform the functions of more than one of the claimed first, second, third or fourth components. Conversely, a plurality of separate physical elements working together may perform the functions of one of the claimed first, second, third or fourth components. Similarly, reference to first, second (etc.) method steps does not limit the invention to only separate steps. According to the invention, a single method step may satisfy multiple steps described herein. Conversely, a plurality of method steps could, in combination, constitute a single method step recited herein. In addition, the steps of the method are not necessarily limited to the order in which they are described or claimed herein.

Advantages provided by the invention over the current technology may optionally include: guaranteeing that any device in which the technology is used will be able to automatically and accurately set the internal clock of the device to local time. When applied to a fully integrated glucose meter this invention may have one or more of the following specific advantages: convenience of never having to set time on the device; assurance that the device's internal clock will always be set to correct local time; significant increase in caregiver's confidence in data collected by meter; improved ability to monitor/detect trends in test results; ability to confidently adjust patient medications based on data collected by meter; allows meter to internally process data using methods not currently used in glucose monitors; can provide users with information not currently available on glucose meters; and providing the user with a clear and constant indication of the number of tests performed or remaining in an integrated meter.

According to a first aspect, the present invention provides a medical diagnostic device comprising means for receiving and processing time data transmitted wirelessly.

According to another aspect, the present invention provides a method for operating a medical diagnostic device, the method comprising: receiving and processing time data transmitted wirelessly.

According to a further aspect, the present invention provides a method of operating a blood glucose meter, the method comprising: receiving and processing time data transmitted wirelessly with the blood glucose meter; performing a plurality of tests with the blood glucose meter; correlating a plurality of glucose concentration measurement values derived from the tests with particular points in time or intervals of time during the day to establish a predetermined range of typical glucose measurement values associated with a particular point in time or time interval; and alerting the user when a measured glucose value falls outside of the predetermined range of typical glucose measurements.

According to yet another aspect, the present invention provides a method of monitoring and treating diabetes with the assistance of a blood glucose meter, the method comprising: receiving and processing time data transmitted wirelessly with the blood glucose meter; performing a plurality of tests with the blood glucose meter; correlating glucose concentration measurement values derived from the tests with the particular points in time during the day when they were taken to establish a data set; downloading the data set from the blood glucose meter; deriving trends based on the downloaded glucose concentration values and associated time data; and prescribing treatment based at least in part on the trends.

According to another alternative aspect, the present invention provides a method of monitoring and treating diabetes with the assistance of a blood glucose meter, the method comprising: receiving and processing time data transmitted wirelessly with the blood glucose meter; performing at least one test with the blood glucose meter thereby creating a blood glucose concentration measurement value; correlating the glucose concentration measurement value with the particular point in time during the day when the test was performed; and automatically identifying the glucose concentration measurement value as being associated with either a pre-prandial test or post-prandial test based at least in part on the correlated time associated with the value, or prompting the user to identify the glucose concentration measurement value as being associated with either a pre-prandial test or post-prandial test.

### BRIEF DESCRIPTION OF THE DRAWING FIGURES

The following description of preferred embodiments can be read in connection with the accompanying drawings in which like numerals designate like elements and in which:

FIG. 1 is a schematic illustration of circuitry used to gather and process signals containing time data.

FIG. 2 is a state diagram illustrating methods whereby a diagnostic medical device may process a signal for use in generating accurate time data for use by the device.

FIG. 3 is a schematic illustration of a diagnostic device constructed according to one optional aspect of the present invention.

### DETAILED DESCRIPTION

According to certain aspects, the present invention is aimed at a fully integrated glucose meter. However, the present invention is not be limited to integrated glucose meters because its benefits can also be applied to conventional (non-integrated) glucose meters, and other diagnostic devices where collection of long term data and analysis of trends in data is important. In the description that follows the term “device” is used to collectively refer to the abovementioned devices/implementations of the principles of the present invention.

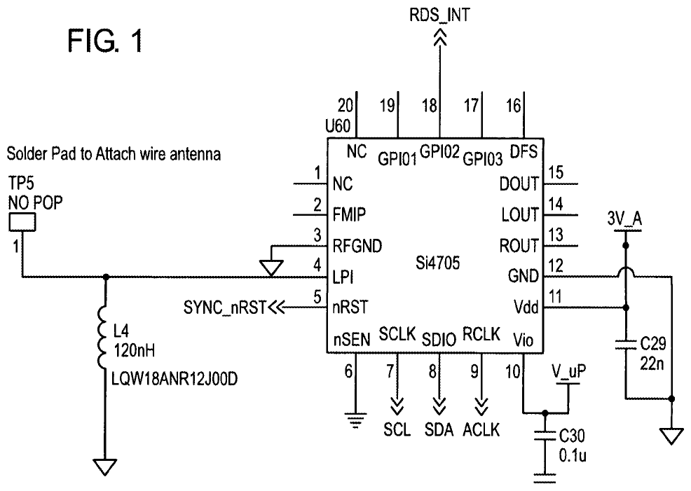

In general terms, when applied to a device, the invention enables the device to use time information commonly available in wireless communications (e.g., radio data system (RDS/RBDS), cell phone, WIFI, etc.) to automatically, quickly, and accurately adjust the internal clock of the device and set it to the correct local time. The invention may include electronic components that when combined give the meter access to time data transmitted wirelessly. Once the correct electronics are in place and the meter can access the information, the data must be carefully processed to ensure that the device is automatically programmed to the correct time. Many electronic components are available that allow access to data in the Radio Broadcast Data Stream (RDS/RBDS). One such component is Si4705 integrated circuit from Silicon Labs, FIG. 1 shows a schematic of such a component, and additional electrical components associated with gathering time data from the RBDS. In addition to the components illustrated in FIG. 1, an antenna may also be provided that allows the integrated circuit to receive RBDS information outdoors and indoors. The antenna can be a simple wire of a specific length optimized to receive RBDS data or any other antenna design to guarantee signal strengths in all intended use environments, homes, offices, cars, parks, etc.

In one alternative embodiment, the integrated circuit and associated electronics are integrated onto the main circuit board assembly of a device.

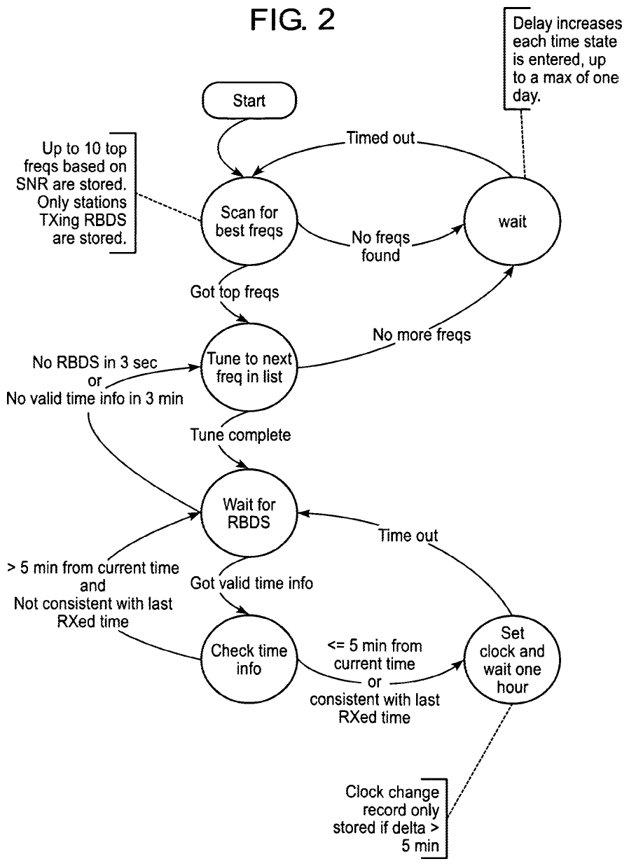

Collecting data is only one aspect of the invention. Once time data is being collected it is processed in a way that allows the device to accurately and efficiently translate the raw radio signal into a usable time. This involves both hardware and software processing of the signal. One optional method of converting a raw RBDS signal into the correct time on a device is illustrated in state diagram form in FIG. 2.

A summary of what is described in the state diagram follows:
1. The device scans local area for a plurality (e.g., up to 10) best RBDS frequencies based on signal to noise ratio (SNR).
   a. Scanning for ten signals allows for redundancy should any signal be lost.
   b. Storing multiple frequencies as a data file also improves the speed at which RBDS time information can be acquired. If a particular frequency does not produce usable time data quickly the meter can change to another frequency without a delay in scanning all frequencies.
   c. Access to multiple signals also allow for a comparison of the time carried by several signals as a verification of having set the correct time on the device.
   d. Searching based on SNR ensures that the device can lock in to the signal sources that are nearest to it, and therefore set the correct time for the device's current position.
2. NOTE: To conserve power the device's firmware may also include instructions to incrementally increase the time between searching for useable frequencies if it encounters difficulty finding a useable signal.
   a. This is illustrated by the “wait” balloon in the top right section of the state diagram.
   b. This algorithm maximizes battery life.
      i. Under certain conditions (shipping, storage, travel to very remote locations, etc.) a useable RBDS may be difficult to locate.
      ii. If the device's program logic was not intelligent enough to determine when to search for a signal it would continue to search until it exhausted its power supply.
3. Once the device has successfully created an internal database of usable RBDS frequencies it begins searching for valid time information.
4. After finding valid time information the device compares the time currently stored in the device with the time carried by the RBDS.
5. If the internally stored time is inconsistent with a verified RBDS time the clock is set to the RBDS time, the device then waits until the next time checking cycle arrives.

Accurately setting the internal clock is only one aspect of the invention. Additional features and benefits that an accurately set clock can provide to a device are also provided by the present invention. The following list is not meant to be exhaustive but is exemplary of some of the potential benefits.

Glucose forecasting will improve the safety of users of an analyte monitor by preventing improper intake of either food or medications.

### Option 1—Use Most Recent Previous Test Data

If the user of a device frequently the device can look at the last few tests to determine if a trend can be identified. For example:
7 pm glucose=125 mg/dL→8 pm glucose=68 mg/dL

The device can identify this as a rapidly falling glucose and may alert the user to test again within a set period of time to verify the trend. It is also possible to suggest the user contact their care provider and/or suggest a potential treatment, such as carbohydrate intake.

### Option 2—Historical Data

If the user of the device consistently tests around the same time period (i.e., upon waking, hour after lunch, etc.) the meter can “learn” the user's typical trends and suggest appropriate actions. For example, if the device consistently detects potential hypoglycemic events (very low glucose values) at a specific time of day, but on a particular occasion detects normal values at the same time the device may encourage the user to test again within a few minutes to verify that the user's glucose levels are normal and not quickly dropping.

The methods and uses of glucose forecasting are not limited to those previously mentioned.

Another use of a reliably accurate internal-clock is the ability to alert users to retest after an abnormal test result. For example if over a defined period of time the user of the device consistently has measured glucose levels within a particular range the device may “learn” the users typical trending. Once the device has learned the user's trends it may begin to expect particular glucose measurements during a particular time of day. If the user completes a glucose measurement that appears abnormal, i.e., out of typical range for time of day, the device may encourage the user to test again to verify the result. While it is possible that the user's glucose measurement may be out of a typical range due to diet, activity levels or response to medication, it also possible that the device may have miscalculated the real glucose value due to a problem with the device. A common benchmark for glucose monitors is the ability to provide glucose measurements that are accurate +/−20% for 90% of the number of tests. This benchmark demonstrates that the potential for the occasional incorrect glucose measurements is widely accepted.

Many people with diabetes are dependent on medications (oral insulin, injectable insulin, or other oral medications) and other treatment plans to maintain their glucose levels within safe and acceptable ranges. For example, a doctor may ask their patient to test immediately upon waking, and then test an hour after breakfast, lunch and dinner. The doctor may then ask their patient to bring their device to their next office visit. When the patient arrives with a device clinical staff may download all of the data that the meter has collected. This data is then analyzed using many methods to suggest appropriate treatment plans. One typical analysis is to plot all of the patient's glucose values compared to time of day. This results in a plot that shows how patient's glucose levels “trend” throughout the day. The clinical staff can then look the patient's trend plot to determine how much medication to prescribe and when to ask their patient to take the medications. This type of analysis can be very effective in helping patient maintain acceptable glucose levels throughout an entire day.

However, complications can arise if the patient's meter does not have correct time information. If the clinical staff notices that the device has the incorrect time they may be able to offset the data and adjust the trend plot so that treatment plans can still be adjusted. This type of adjustment is dependent on knowing how long the device's internal clock has been improperly set. For example, it would be completely unknown to the clinical staff if the patient's device had correct time for half the results, but then incorrect time for the second half of the results. The clinical staff seeing the incorrect time may then shift every result so that half of them are still incorrect. At best, the clinical staff may decide not to change treatment plans due to lack of confidence in the data. At worst, the clinical staff may incorrectly adjust the treatment plan and unknowingly put the patient at risk.

It is also possible that the clinical staff may not notice that the device has the incorrect time set and may read an incorrect trend plot and adjust the treatment plan incorrectly. Again, this can prevent a patient from improving their disease management or even put the patient at risk for over or under medicating.

Many patients with diabetes receive their treatments at either endocrinologists, or primary care doctors focused on working with these patients. The offices that treat patients with diabetes can therefore see several patients in a given day. To maintain an efficiently running office clinical staff need to quickly be able to collect information from patient's device, process the information, and provide data to the clinical staff to allow them to determine effectiveness of current treatments and create modified treatment plans to improve patient's disease management. The process of collecting data from patient's device is already very time and labor intensive. Each device currently on the market has its own individual data transfer cable and data transfer software. When a meter is processed for data download the clinical staff needs to identify the correct cable, the correct software and then hope that the transfer occurs without any issues. If the clinical staff is distracted by trying to figure out how to properly set the time on a device, valuable time will be wasted. Since every device on the market has an individual user interface, it is not uncommon that the clinical staff would have to look up how to adjust the device's clock in a users guide, wasting even more time. This invention ensures that patient's device is always set correctly and therefore will directly save time and money for clinics providing care for patients with diabetes.

Another way in which this invention provides a time and cost savings for clinics is by speeding up the training that each patient must get when receiving a new device. Currently, it is common practice for clinical staff (Certified Diabetes Educator, etc.) to spend time training each patient when they receive a new device. Such training typically includes basic meter operation, and some time spent setting up the device (user preferences, time of day, etc.). The removal of training for setting the clock will provide an instant time savings. This time saving may seem minimal, but when multiplied by the thousands of patients treated by a clinic every year it is very significant.

One of the key factors that clinical staff monitor in their patients while creating and maintaining treatment plans is the patient's glucose levels before and/or 1-2 hours after eating; these tests are typically called pre-prandial and post-prandial tests. This data can be extracted manually from the data collected by the device by looking at the time a test occurred and assuming it was before or after a particular meal. Another way of collecting this data relies on a feature that some devices have that allow user's to mark a particular test as either pre- or post-prandial. There are deficiencies in both methods.

If the data is manually extracted, the clinical staff must first assume that the time on the device was correct when the data was collected, then be comfortable assuming that data collected during a particular time was either pre or post-prandial, and also be able and willing to dedicate more time to a labor intensive data extraction process.

Data marked by the users of the device can be more accurate, however, this method only works if a user understands how to mark a test, selects the appropriate meal marker, i.e., pre- or post-breakfast, etc., remembers to mark all of their tests so that the averages created can have an accurate and sufficiently large data set.

Another benefit of this invention is that it enables a device to accurately and automatically mark tests as either pre- or post-prandial. The following is one of the many possible logic sequences or operational modes that will allow a device to automatically meal stamp a test.
1. The device automatically sets and maintains internal clock as previously described.
2. Option 1: The device can be automatically setup to group tests as pre- or post-meal depending on time of day
   a. Ex. Tests between 5-7 am are always marked as pre-breakfast and tests between 9-10 am are always marked as post-breakfast, etc.
3. Option 2: User can setup the device manually so that it “learns” when they have particular meals and use this data to automatically mark tests.
   a. Ex. User programs device once to know that tests between 6-8 am are pre-breakfast and tests between 9-11 am are post-breakfast, etc.
4. After a user completes a glucose measurement the device can compare the test's timestamp against the internal registry for pre- or post-meal times.
5. If a test falls within one of the meal time windows the device can automatically add a pre- or post-meal marker.
6. Optional: The device can automatically ask the user to verify that the test was pre- or post-meal. This is redundant but can add even more confidence to the dataset and will not require the user to navigate a confusing menu. A simple yes/no prompt is sufficient.
7. Optional: The device can store a continuously updating average of each pre- or post-meal glucose values.
   a. This average can be quickly accessed using a simple user interface directly on the device.
   b. Easy access to the data allows clinical staff to make well-informed treatment decisions without needing access to computers, cables, or additional software.

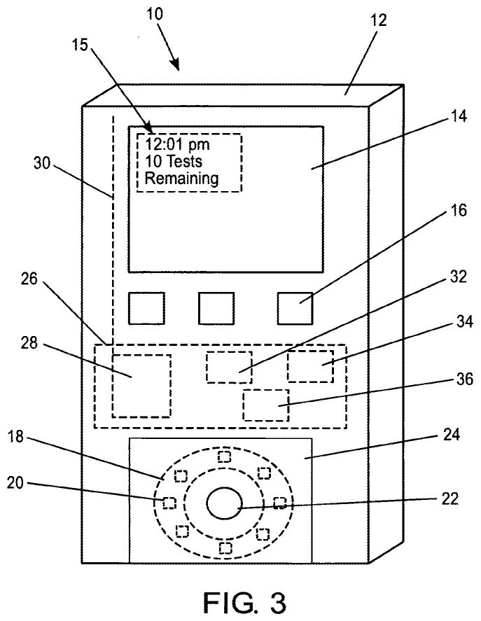

A device formed according to further optional aspects of the present invention is illustrated in FIG. 3. The device 10 may comprise a housing 12 of any suitable form, formed of any suitable material. The device comprises a display 14. The device 10 can be provided with enhanced user interface functionality by providing a visual and/or audible signal of at least one of: the number of tests performed; and the number of test remaining to be performed, by the device. The device 10 may provided with one or more interface devices, such as buttons 16.

As alluded to above, a device 10 in the form of an integrated meter includes all of the necessary components for performing an assay to determine the concentration and/or presence of a target analyte. An integrated meter may in fact, according to the principles of the present invention, be provided with the necessary components for performing a plurality of tests, each capable of determining the presence and/or concentration of a target analyte substance. For example, an integrated meter may optionally be provided with a removable cartridge 18 or similar device which contains the necessary components 20 for performing a plurality of tests. The device 10 may comprise a test port 22 for accessing the components 20 of the cartridge 18 to perform a test. The cartridge may be removed through a door or cover 24 forming part of the housing 12. The number of tests which have been performed, or which are available for use before the cartridge 18 must be replaced, becomes important information to communicate to the user of such devices. According to the present invention, an integrated meter may be provided with the means necessary to produce a suitable audible and/or visual signal to the user to convey such information. Any suitable signal may be utilized. Suitable audible signals may include spoken information concerning the number of test performed and/or remaining. Suitable visual signals may include alphanumeric characters which specify the number of tests performed and/or remaining 15. These visual signals 15 may be continuously displayed by the integrated meter, even when the meter itself is “powered down” or otherwise shut off or placed in a “sleep” mode. According to a further alternative, the integrated meter may be designed such that it is inverted or flipped when it is to be applied to the skin of a user. Thus, according to a further aspect of the present invention, the device may include a suitable means for sensing is inverted or flipped condition and the visual display of alphanumeric characters may likewise be flipped or inverted so as to facilitate reading by the user, regardless of the orientation of the integrated meter.

The device 10 may further comprise a circuit board 26 comprising electronic components. These components may include components for receiving and processing time data transmitted wirelessly, as previously described herein Thus, the device 10 may comprise any of the functionality previously described herein. An integrated circuit 28 for receiving, and optionally at least partially processing, wirelessly transmitted time data may be provided. The integrated circuit 28 may take any suitable form, such as the Si4705 circuit commercially available from Silicon Labs, as previously described herein. The integrated circuit 28 may be provided with a suitable antenna 30, such as a wire-like antenna contained within the housing 12 of the device 10. Other suitable antennas are envisioned, such as external flexible antennas, and the like. The circuit board may further include a number of additional electronic components, such as a processor 32, memory 34 and power supply 36. The processor 32 may optionally cooperate with the integrated circuit 28, and may also process time data received thereby for use by the device 10.

Numbers expressing quantities of ingredients, constituents, reaction conditions, and so forth used in this specification are to be understood as being modified in all instances by the term “about”. Notwithstanding that the numerical ranges and parameters setting forth, the broad scope of the subject matter presented herein are approximations, the numerical values set forth are indicated as precisely as possible. Any numerical value, however, inherently contains certain errors necessarily resulting from the standard deviation found in their respective measurement techniques. None of the elements recited in the appended claims should be interpreted as invoking 35 U.S.C. § 112, 16, unless the term “means” is explicitly used.

Although the present invention has been described in connection with preferred embodiments thereof, it will be appreciated by those skilled in the art that additions, deletions, modifications, and substitutions not specifically described may be made without departing from the spirit and scope of the invention as defined in the appended claims.

1. A method of operating a blood glucose meter, the method comprising:
receiving and processing time data transmitted wirelessly with the blood glucose meter;
performing a plurality of tests with the blood glucose meter;
correlating a plurality of glucose concentration measurement values derived from the tests with particular points in time or intervals of time during the day to establish a predetermined range of typical glucose measurement values associated with a particular point in time or time interval; and
alerting the user when a measured glucose value falls outside of the predetermined range of typical glucose measurements.

2. A method of monitoring and treating diabetes with the assistance of a blood glucose meter, the method comprising:
receiving and processing time data transmitted wirelessly with the blood glucose meter;
performing at least one test with the blood glucose meter thereby creating a blood glucose concentration measurement value;
correlating the glucose concentration measurement value with the particular point in time during the day when the test was performed; and
automatically identifying the glucose concentration measurement value as being associated with either a pre-prandial test or post-prandial test based at least in part on the correlated time associated with the value, or prompting the user to identify the glucose concentration measurement value as being associated with either a pre-prandial test or post-prandial test.

3. The method of claim 2, wherein the blood glucose meter comprises predefined time intervals associated with either a pre-prandial test or post-prandial test.

4. The method of claim 2, further comprising programming the blood glucose meter to define the pre-prandial test and post-prandial test time intervals.

5. A method of monitoring and treating diabetes with the assistance of a blood glucose meter, the method comprising:
receiving and processing time data transmitted wirelessly with the blood glucose meter;
performing a plurality of tests with the blood glucose meter;
correlating glucose concentration measurement values derived from the tests with the particular points in time during the day when they were taken to establish a data set;
downloading the data set from the blood glucose meter;
deriving trends based on the downloaded glucose concentration values and associated time data; and
prescribing treatment based at least in part on the trends.

6. A method for operating a medical diagnostic device, the method comprising:
receiving and processing time data transmitted wirelessly.

7. The method of claim 6, wherein receiving and processing time data comprises scanning the local area for wireless time data transmissions, calculating a signal-to-noise ratio for each wireless time data transmission received, and comparing the signal-to-noise ratio of a plurality of wireless time data transmissions received by the device.

8. The method of claim 6, wherein receiving and processing time data comprises storing the plurality of frequencies as a data file.

9. The method of claim 6, wherein receiving and processing time data comprises automatically and incrementally increasing the time between scanning the local area for wireless time data transmissions upon encountering difficulty in acquiring signals of a predetermined acceptable quality.

10. The method of claim 6 wherein receiving and processing time data comprises comparing time data currently stored within the device with time data obtained from at least one wireless time data transmission.

11. The method of claim 10, wherein the means for receiving and processing time data further comprises overwriting the time data currently stored within the device with time data obtained from at least one wireless time data transmission upon detection of a discrepancy therebetween.

12. The method of claim 6, wherein the time data is transmitted via airwaves.

13. The method of claim 12, wherein the time data is transmitted from at least one of the following sources: radio broadcast data stream (RBDS); global positioning system (GPS); wireless fidelity network (WiFi); and the National Atomic Clock signal.

14. A medical diagnostic device comprising a wirelessly transmitted time data receiver and processor.

15. The device of claim 14, wherein the medical diagnostic device comprises an integrated glucose meter.

16. The device of claim 14, further comprising an antenna.

17. The device of claim 14, wherein the receiver and processor comprises an integrated circuit.

18. The device of claim 14, wherein the receiver and processor are configured for scanning the local area for wireless time data transmissions, calculating a signal-to-noise ratio for each wireless time data transmission received, and comparing the signal-to-noise ratio of a plurality of wireless time data transmissions received by the device.

19. The device of claim 18, wherein the receiver and processor are configured for storing the plurality of frequencies as a data file.

20. The device of claim 18, wherein the receiver and processor are configured for automatically and incrementally increasing the time between scanning the local area for wireless time data transmissions upon encountering difficulty in acquiring signals of a predetermined acceptable quality.

21-24. (canceled)
